(19) United States
(12) Patent Application Publication
(10) Pub. No.: US 2005/0101560 A1
(43) Pub. Date: May 12, 2005

(54) Method of treating an individual with methyl 1-[N6-(3-iodobenzyl)-adenin-9-yl] beta-D-ibofuronamide

(75) Inventors: Steve Warrington, Middlesex (GB); Michael Silverman, Marblehead, MA (US); William Kerns, Harvard, MA (US); Pnina Fishman, Herzliya (IL); Ilan Cohn, Herzliya (IL)

Correspondence Address: BROWDY AND NEIMARK, P.L.L.C.;624 NINTH STREET, NW, SUITE 300, WASHINGTON, DC, 20001-5303, US

(73) Assignee: CAN-FITE BIOPHARMA LTD., Petach Tikva (IL)

(21) Appl. No.: 10/705,262

(22) Filed: Nov. 12, 2003

(51) Int. Cl. A61K 31/7076

(52) U.S. Cl. 514/46

(57) ABSTRACT

A method for treating an individual with IB-MECA to achieve a therapeutic effect is provided. The method comprises administering to the individual a dose of IB-MECA in an amount and for a time such so as to achieve a maximal blood level of less than about 1.60 nM.

### FIELD OF THE INVENTION

The present invention is generally in the field of human therapy and concerns the pharmaceutical use of methyl 1-[N6-(3-iodobenzyl)-adenin-9-yl]-β-D-ibofuronamide (known in the art and will also be referred to herein as IB-MECA).

### PRIOR ART

The following prior art is considered to be pertinent as background to the present invention:
1. Jacobson et al., U.S. Pat. No. 5,773,423
2. Fishman P., Bar-Yehuda S., Ohana G., Pathak S., Wasserman L., Barer F., Multani A. Eur J. Cancer 36: 1452-58, 2000a.
3. Bar-Yehuda S, Barer F, Volfsson L, Fishman P. Resistance of muscle to tumor metastases: A role for A3 adenosine receptor agonists. Neoplasia 3: 1-7, 2001.
4. Fishman P, Bar-Yehuda S, Rath-Wolfson L, Ardon E, Barrer F, Ochaion A, Madi L. Targeting the A3 adenosine receptor for cancer therapy: inhibition of prostate carcinoma cell growth by A3AR agonists. Anticancer Res. In Press, 2003.
5. Bar-Yehuda S, Madi L, Barak D, Mittelman M, Ardon E, Ochaion A, Cohn S, Fishman P. Agonists to the A3 adenosine receptor induce G-CSF production via NF-kappaB activation: a new class of myeloprotective agents. Exp. Hematol. 30:1390-8, 2002.
6. Fishman P, Bar-Yehuda S, Farberstein T, Barer F, Ohana 0. Adenosine acts as a chemoprotective agent by stimulating G-CSF production: a role for A1 and A3 adenosine receptors. J Cell Physiol 183: 393-8, 2000b.

### BACKGROUND OF THE INVENTION AND PRIOR ART

IB-MECA is selective A3 adenosine receptor agonist (Jacobson et. al, U.S. Pat. No. 5,773,423). It was shown that IB-MECA has a cytostatic effect on various tumour cell types by arresting cell growth at GO/GI phase of the cell cycle (Fishman et al, 2000a). In vivo, orally-administered IB-MECA inhibits the development of tumours in syngeneic (melanoma, colon carcinoma) and xenograft (colon and prostate carcinoma) mouse models (Bar-Yehuda et al, 2001; Fishman et al, 2003). Administering IB-NECA orally to mice stimulates the production of neutrophils via an increase in G-CSF (Bar-Yehuda et al, 2002; Fishman et al, 2000b) and thus when administered with chemotherapy, IB-MECA protects against myelotoxicity. Moreover, oral administration of IB-MECA inhibits colon carcinoma growth in nude mice, and stimulates neutrophil recovery after cytotoxic drg therapy (Bar-Yehuda et al, 2001; Fishman et al, 2003).

In past animal studies carried out with RB-MECA the administered doses spanned 4 orders of magnitude ranging between 10 μg/Kg (Fishman et al, 2000a; Fishman et al, 2000b; Bar-Yehuda et al, 2001; Bar-Yehuda et al, 2002; Fishman et al, 2003) and 100 mg/Kg.

Up to now, IB-MECA was tested only on laboratory animals.

### SUMMARY OF THE INVENTION

I the following description and claims, the term “individual” refers to a human individual.

In accordance with the invention a clinical trial was conducted that permitted for the first time to determine the maximal administered dose that can be administered without causing side effects. Healthy adult individuals received IB-MECA orally and pharmacodynamic effects of this drug were examined. It was found that a single daily oral dose of IB-MECA up to 5 mg was safe while at a daily dose of 10 mg some cardiovascular-related side effects were seem A twice daily oral dose (given to the individuals at 12 hour interval) of 4 mg (total daily dose of 8 mg) was also found to be safe, while a dose of 5 mg given twice daily was found to induce some cardiovascular-related adverse events.

The side effects were found to be correlated with a blood level of IB-MECA exceeding about 160 nM (about 80 ng/ml, given IB-MECA's molecular weight of 510 daltons).

The present invention thus provides a method for treating an individual with IB-MECA to achieve a therapeutic effect, the method comprises administering to the individual a dose of IB-MECA in an amount and for a time such so as to achieve a maximal blood level of less than about 160 nM.

The dose of administered IB-MECA to achieve a blood level in accordance with the invention can be easily gauged in a clinical study such as that described bellow. In such a study individuals may be given IB-MECA at different dosages and blood samples may be taken in some time points and thereby the dose that yields a blood level of IB-MECA of 160 nM can be determined. As will no doubt be appreciated, this absolute dose of IB-MECA will be different depending on the route of administration. For example, it is expected that intravenously administered IB-MECA will yield a blood level of 160 nM at a lower administered dose than following oral administration (as in the case of the latter, the achieved blood level is limited by absorption through the digestive tract. Also, the total dose may dependent on factors such as age, gender, general health condition, etc. For example, it is expected that the total dose in children that will yield a blood level of 160 nM could be more or less than in adults.

A preferred route of administration is oral. However, the invention is not limited to oral administration and RB-MECA may be administered in any one of a number of administration routes that are currently acceptable or that will become acceptable in the future such as nasal tansdermal, parenteral, rectal, etc. For oral administration IB-MECA may be formulated as a drink or syrup, may be formulated in the form of pills, capsules or lozenges, etc. As IB-MECA is not water soluble, a liquid formulation may require the use of emulsifiers, surfactants, etc., in order to keep IB-MECA in the solution.

In accordance with an embodiment of the invention, the treated individuals are adults, the administration rote is oral. In accordance with this embodiment, the dose of IB-MECA for a single daily dose is less than about 5 mg and the dose of IB-NECA for twice daily dosing is less than 4 mg each dose (less than 8 mg total daily dose). A preferred dose of IB-MECA, in accordance with this embodiment is in the range of about 0.1 to about 5 mg for a single daily administration; a preferred dose of IB-MECA for twice daily administration in the range of about 0.1 to about 4 mg (i.e. 0.2 to 8 mg total daily dose).

The present invention, also provides, in accordance with the aforementioned embodiment, a pharmaceutical composition in a dosage form, comprising a pharmaceutically acceptable carrier and IB-NECA at an amount for administration of IB-MECA of up to about 5 mg in a single daily dose or up to about 4 mg for twice daily administration. It should be clear to the artisan that the dosage form may comprise less than the desired administration dose such that an individual may need to take 2 or 3, etc. dosage forms of the composition to achieve his needed dose. Thus, by way of an illustrative example only, if the intended dose is 2 mg of IB-MECA, the dosage form may include 0.5 mg, 1 mg or 2 mg of IB-MECA and the individual will then need to take 4, 2 or 1 such dosage forms, respectively, to achieve the desired dose.

The method and pharmaceutical composition of the invention are useful for the treatment of diseases or disorders, that can be cured or ameliorated by the administration of an A3 adenosine receptor agonist to a needing subject. Examples are: treatment of malignancies, particularly solid tumors, in order to arrest tumor growth; treatment of rheumatoid arthritis in order to reduce the inflammatory response; treatment of subjects suffering from neutropenia in order to boost up their neutrophil count; treatment of subjects with risk of cardiac or neural ischemia; and others.

The invention will be further illustrated bellow with reference to the specific embodiment concerned with oral administration. Described bellow is a clinical study that was done to determined the maximal dose in adult individuals. It will no doubt be appreciated by the artisan that this is an example and this description of the specific embodiment is not intended to be limiting to the full scope of the invention as defined above and in the appended claims.

### BRIEF DESCRIPTION OF THE DRAWINGS

FIG. 1 shows the change in semi-recumbent heart rate as a function of plasma IR-MECA concentration after single doses of IB-MECA.

FIG. 2 shows the Change in standing heart rate as a function of plasma MB-MECA concentration after repeated doses of IB-MECA.

### DESCRIPTION OF SPECIFIC EMBODIMENT

Methods

Study design

Two clinical studies were carried out: a single dose study and a repeat dose study. Both studies were parallel-group, double-blind, dose-rising, and placebo-controlled in design.

In the single dose study, 15 healthy men (3 groups of 5) received a single oral dose of IB-MECA (1, 5 or 10 mg) or placebo. In each group, 1 subject received placebo. In the repeated dose study, 28 healthy men (4 groups of 7) received repeated oral doses of IB-MECA (2, 3, 4 or 5 mg) or placebo every 12 h for 7 days. In each group, 2 subjects received placebo.

Selection of Subjects

Healthy young men, aged 18-45 years.

Study Drugs

In the single dose study, a solution with IB-MECA powder in 30% Cremophor RH40 (BASF) was used. In the repeated dose study, an aqueous 0.5% methylcellulose suspension (Methocel A4M Premium, The Dow Chemical Company) was used. In both studies, the study medication was taken orally as a drink, followed by 50 ml of tap water.

Study Procedures

The following procedures were done:
Safety assesment: laboratory assessments (routine biochemistry and urinalysis), physical examination, 12-lead ECO, ambulatory ECG, pulmonary function testing (FEV1), vital signs (semi-recumbent in the single dose study; semi-recumbent and standing in the repeat dose study). Adverse events were recorded throughout both studies.
Determination of IB-MECA blood level: in the single dose study blood samples for assay of IB-MECA were taken immediately before and at 0.25, 0.5, 1, 2, 4, 8, 12, 24, and 48 h after dosing; in the repeat dose study—blood samples were taken immediately before and at 0.25, 0.5, 1, 2, 4, 8, and 12 h after dosing on Day 1, before dosing on Days 26, and before and at 0.25, 0.5, 1, 2, 4, 8, 12, and 24 h after dosing on Day 7.

Plasma samples were assayed for IB-MECA using LC/MS/MS. The lower limit of quantification (LLOQ) was 0.1 ng/mL. Intra-assay coefficients of variation (CV) were <5.0% and inter-assay CVs were <9.4%.

Pharmacokinetic Analysis

Maximum concentration (Cₘₐₓ) and time to maximum concentration (tₘₐₓ) were observed values. Other pharmacokinetic parameters (half-life, to AUC; and clearance, CL/F) were calculated by non-compartmental methods using WinNonlin® software (version 3.0, Pharsight, Mountain View, Calif., US). Accumulation indices of Cₘₐₓ and AUC were calculated as ratio of values at steady state (Day 7) to the values on Day 1.

Statistical Analysis

Data from all subjects who received IB-MECA were included in the analysis of safety and tolerability (adverse events and laboratory safety variables). Numerical data and parameters were summarised using means or medians, and other descriptive statistics, according to the type and distribution of the data.

Results

Study Population

In the single dose study, the mean (range) age, weight and height were 28.3 (2040 years, 75.9 (63-98) kg, and 177.8 (167-188) cm, respectively. In the repeat dose study, the mean (range) age, weight, and height were 25.2 (18-45) years, 75.3 (56-99) kg, and 178.0 (163-189) cm, respectively. All volunteers were of Europid ethnic origin, except for 2 Asian/Indian men and 1 Europid/Oriental man.

All subjects were deemed healthy at screening, without any haematological disorder or history of splenectomy, nor splenomegaly on physical examination.

Safety and Tolerability

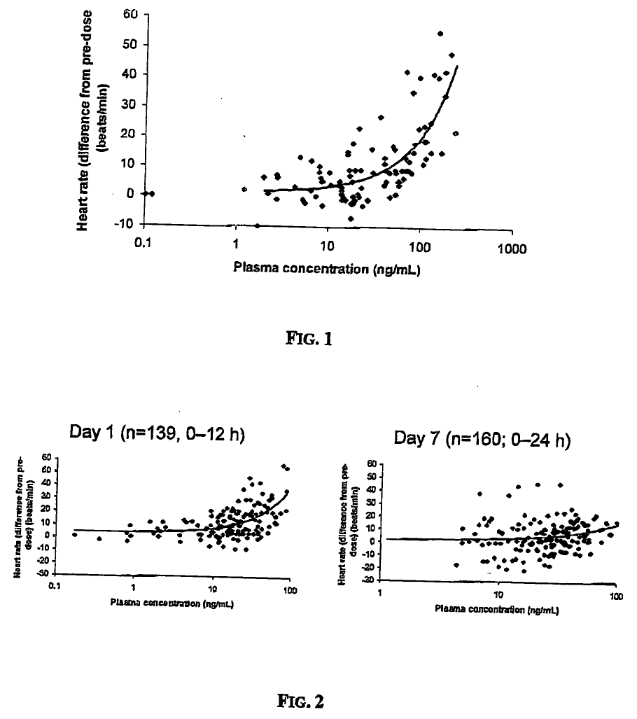

In the single dose study, IB-MECA in doses up to 5 mg was well tolerated, as judged by vital signs, physical examination, FEV1, and 12-lead and continuous ECG. There were no clinically significant changes in safety tests of blood and urine. Four subjects had a small increase in resting heart rate after 5 mg IB-MECA; however, after the 10 mg dose, 4 subjects had substantial increases in resting heart rate, 2 of which were substantial (up to 115 beats/min) and considered drug-related. Those 2 subjects developed nausea, and 1 of them vomited once and was facially flushed. Those changes precluded our studying higher doses. In no subject was there a significant change in blood pressure, but blood pressure was not measured in the standing position. The increase in heart rate was closely related to the plasma IB-MECA concentration (FIG. 1).

In the repeat dose study, IB-MECA had an acceptable safety profile, as judged by vital signs, physical examination, FEV1, and 12-lead and continuous ECG. There was a dose-related increase in heart rate on Day 1, but some tolerance developed, because that effect was clearly smaller on Day 7. On Day 1, the time course of the increase in heart rate reflected the profile of plasma IB-MECA concentrations. However, on Day 7, equivalent plasma IB-MECA concentrations were associated with smaller increases in heart rate (FIG. 2). There were no clinically significant changes in safety tests of blood and urine.

Most of the adverse events occurred during the 5 mg dose regimen: headache and drowsiness were most common. Two adverse events were vascular disorders—hot flushes and dizziness on standing.

Overall, 13-MCA was well tolerated at single doses of up to 5 mg and repeat doses of up to 4 mg 12-hourly. Adverse events were related to dose and generally occurred around the time of maximal blood concentration (t. After single doses of up to 5 mg IB-MECA, there were no adverse events within 12 h of dosing, but after a single dose of 10 mg, there were 8 adverse events within 12 h of dosing. After repeated doses of up to 4 mg 12-hourly, there were 2 adverse events within 12 h of dosing. However, after repeated doses of 5 mg 12-hourly, there were 13 adverse events within 12 h of dosing. Thus, based on this repeat dose study, the 4 mg dose was determined to be the maximum tolerated dose for a twice-daily therapeutic regimen.

Overall a single daily dose of 5 mg and a twice daily dose of 4 mg where considered safe and well tolerated. Given the fact that these doses gave a plasma level (Cmax) of less than 160 nM (80 ng/ml)

Pharmacokinetics

The pharmacokinetics of single doses of IB-MECA are shown in the following Table 1:

TABLE 1
Mean (SD) plasma PK parameters after a single
oral dose of CF101 (n = 4 per group)

| Dose | Cₘₐₓ | tₘₐₓ^{a} | AUC₍₀₋₄₈₎ | AUC_{(inf)} | t_{1/2} | CL/F |
|---|---|---|---|---|---|---|
| (mg) | (ng/mL) | (h) | (ng · h/mL) | (ng · h/mL) | (h) | (L/h) |
| 1 | 21.2 (2.1) | 1 (1-2) | 220.7 (20.9) | 225.2 (21.7) | 8.7 (0.7) | 4.5 (0.4) |
| 5 | 81.6 (23.6) | 1 (1-2) | 872.3 (211.6) | 904.0 (221.9) | 8.3 (0.2) | 5.8 (1.4) |
| 10 | 178.0 (46.6) | 1 (1-2) | 1780.0 (228.7) | 1813.0 (226.5) | 8.6 (0.4) | 5.6 (0.7) |

^{a}median (range)

As can be seen, IB-MECA pharmacokinetics were linear, and inter-subject variability was low. IB-MECA was absorbed rapidly: tₘₐₓ ranged between 1-2 h. Mean Cₘₐₓ (maximal plasma level) and AUC₀₋₄₈ (area under the curve of blood level over 48 hours after administration) were related to dose. Cₘₐₓ was 21.2, 81.6, and 178.0 ng/ml, and AUC₀₋₄₈ was 220.7, 872.3, and 1780.0 ng.h/ml, for doses of 1, 5, and 10 mg, respectively. The half-life of about 8.5 h was independent of dose. Apparent plasma clearance (CL/F) was low (47 L/h) and independent of dose.

The pharmacokinetics of repeated doses of IB-MECA are shown in the following fable 2:

TABLE 2
Mean (SD) plasma PK parameters on Days 1 and 7 of
repeated dosing with CF101 (n = 5 per group)

| Dose | | Cₘₐₓ | tₘₐₓ^{a} | AUC₍₀₋₁₂₎ | AUC^{b} | t_{1/2} | CL/F |
|---|---|---|---|---|---|---|---|
| (mg) | Day | (ng/mL) | (h) | (ng · h/mL) | (ng · h/mL) | (h) | (L/h) |
| 2 | 1 | 22.0 (3.3) | 2 (1-4) | 155.2 (39.1) | 207.4 (52.0) | 5.52 (0.2) | 10.1 (2.3) |
| | 7 | 30.9 (3.1) | 2 (1-2) | 242.4 (41.4) | 346.3 (64.0) | 9.83 (1.2) | 4.9 (0.7) |
| 3 | 1 | 49.3 (9.7) | 2 (1-2) | 304.5 (19.5) | 423.5 (27.5) | 6.29 (1.0) | 7.1 (0.5) |
| | 7 | 49.0 (7.9) | 1 (1-2) | 341.6 (38.1) | 512.1 (74.1) | 9.25 (0.8) | 5.0 (0.6) |
| 4 | 1 | 46.2 (11.4) | 2 (1-2) | 297.0 (57.2) | 400.2 (85.7) | 5.77 (0.6) | 10.4 (2.1) |
| | 7 | 58.1 (10.4) | 1 (1-2) | 458.3 (54.8) | 640.3 (73.7) | 8.93 (0.8) | 5.4 (0.7) |
| 5 | 1 | 63.6 (22.0) | 2 (1-2) | 461.6 (157.4) | 596.1 (196.6) | 4.96 (0.3) | 9.3 (3.5) |
| | 7 | 79.5 (24.1) | 2 (2-2) | 601.0 (163.6) | 818.4 (214.0) | 9.39 (0.6) | 5.4 (1.5) |

^{a}median (range)
^{b}AUC_{(inf)} on Day 1, AUC₍₀₋₂₄₎ on Day 7

IB-MECA was absorbed rapidly: tₘₐₓ was 1-2 h. Steady state was reached by Day 3. IB-MECA pharmacokinetics did not change after repeated dosing. Plasma concentrations of IB-MECA were dose proportional on Day 1 and at steady state (Day 7). Half-life of IB-MECA was independent of dose, and was about 9-10 h at steady state. As in the single dose study, apparent plasma clearance (CL/F) was low (5-10 L/h) and independent of dose. The accumulation indices ranged between 1-1.4 and 1.1-1.6 for Cₘₐₓ and AUC, respectively; the accumulation indices were much as predicted from the single dose data.

Summary

Overall a single daily dose of 5 mg and a twice daily dose of 4 mg where considered safe and well tolerated. Given the fact that. These doses gave a Cₘₐₓ of less than about 160 nM (80 ng/ml).

1. A method for treating an individual with IB-MECA to achieve a therapeutic effect, the method comprises administering to the individual a dose of IB-MECA in an amount and for a time such so as to achieve a maximal blood level of less than about 160 nM.

2. A method according to claim 1, wherein IB-MECA is administered orally.

3. A method according to claim 2, wherein IB-MECA is administered to an adult individual in a single daily dose of less than about 5 mg.

4. A method according to claim 1, wherein IB-MECA is administered at a dose of about 0.1 to about 5 mg.

5. A method according to claim 2, wherein IB-NECA is administered to an adult individual in two daily doses of less than about 4 mg each dose.

6. A method according to claim 5, wherein IB-MECA is administered at a dose of about 0.1 to about 4 mg each dose.
